(19) United States
(12) Patent Application Publication
(10) Pub. No.: US 2024/0198018 A1
(43) Pub. Date: Jun. 20, 2024

(54) Insufflation Tube for Laparoscopy Comprising Heating Element, Humidifying Material and Device for Determining the Moisture Content

(71) Applicant: W.O.M. WORLD OF MEDICINE GMBH, Berlin (DE)

(72) Inventor: Bernd Müller, Berlin (DE)

(73) Assignee: W.O.M. WORLD OF MEDICINE GMBH, Berlin (DE)

(21) Appl. No.: 18/558,109

(22) PCT Filed: May 2, 2022
(86) PCT No.: PCT/IB22/54025

§ 371 Date: Oct. 30, 2023

(30) Foreign Application Priority Data
Apr. 30, 2021 (DE) 10 2021 002 272.6
Jul. 7, 2021 (DE) 10 2021 003 506.2

(51) Int. Cl. A61M 13/00; A61M 16/10

(52) U.S. Cl. CPC A61M 13/003; A61M 16/109; A61M 16/1095; A61M 2205/18; A61M 2205/3368

(57) ABSTRACT

The present invention relates to an insufflator having an insufflation tube comprising an integrated heating element and humidifying material for laparoscopy, wherein measurement of the moisture content is possible. No separate humidity sensor is required to measure the moisture content of the humidifying material. Components that are already part of the insufflation tube can be used, in particular, the lead wires of heating elements or temperature sensors.

### CROSS-REFERENCE TO RELATED APPLICATIONS

This application is a 35 U.S.C. § 371 National Stage filing of International Application No. PCT/IB2022/054025, filed on May 2, 2022, which patent application claims priority from German Patent Applications No. 102021002272.6 filed on Apr. 30, 2021 and German Patent Applications No. 102021003506.2 filed on Jul. 7, 2021 the entire disclosure of each is incorporated herein by reference.

### BACKGROUND OF THE INVENTION

### 1. Field of the Invention

The present invention relates to an insufflator having an insufflation tube comprising an integrated heating element and humidifying material for laparoscopy, whereby measurement of the moisture content is possible.

### 2. Background and Prior Art

Laparoscopy is a medical procedure that allows visual inspection of the abdominal cavity and the organs inside. Typically, small skin incisions (0.3 to 2 centimeters) are usually made in the abdominal wall and a trocar is inserted through the incision, which in turn can accommodate an optical device. A special endoscope (laparoscope) is used to view the abdominal cavity. In diagnostic laparoscopy, the abdominal cavity is only visually inspected; in therapeutic laparoscopy, surgical procedures may also be performed.

Typically, laparoscopy begins by filling the abdominal cavity with gas to create a pneumoperitoneum. Various gases have been used, including air, nitrogen, and carbon dioxide (CO2). The use of carbon dioxide gas has been particularly successful. It has been found that it is useful to heat and humidify the gas introduced, especially during longer laparoscopic procedures. Heating the gas is used to prevent the patient from cooling down and to avoid a diffuse pain sensation in the patient, which is probably a consequence of local cooling due to the entry of cold gas. Humidification is used to prevent the internal abdominal surfaces from drying out and cooling down.

Proposals for this have already been made in the prior art. For example, the German patent specification DE 19510710 describes a device that includes a means for adjusting the gas humidity (e.g., a sponge) and may optionally include an additional heating element.

DE 10 2013 000492 A1 describes a tube with an integrated heating element for laparoscopy, which also contains a humidifying material. According to this document, the humidifying material is humidified with water prior to an operation. Depending on the water absorption of the material described there, the gas volume flow and the duration of the surgery, it may be necessary to re-humidify the humidifying material intraoperatively. Since the rate of water evaporation depends on a number of parameters, it is only possible to estimate when replenishment is possible. Alternatively, designs have been described that use a humidity sensor to determine the gas humidity in the gas channel. However, this has several disadvantages. First, the humidity sensor must be electrically connected, which complicates the design of the filter interface. In addition, the humidity sensor creates a significant flow resistance in the gas channel. This results in a lower flow rate, which is contrary to today's flow requirements.

Another device for humidifying gases in medical technology is described in DE 3617031A1 (priorities: NZ 21263, NZ 215123, and NZ 214694). In the context of a tube system which has to be manufactured in a complex way, a tube is provided which is always filled with water. Water vapor is released to the gas through a microporous tube wall. A sensor monitors the temperature of the water.

Another solution known in the prior art is to use a humidity sensor. One such solution can be a sensor that measures the temperature curve during heating, as described in WO 2017/157 365 A1.

Another method of determining the residual humidity without a humidity sensor is described in U.S. Pat. No. 8,836,521 “Hydration Alert”: The heat work (electrical work) performed is determined and used to trigger an alert to the user to replenish the humidifying medium. It is also described how to use the total amount of insufflated expansion medium to trigger a refill alarm. The disadvantage of the solutions described is the high cost of sensors and equipment in the device, combined with only an indirect determination of the humidifying medium present in the humidifying braid.

EP 2388041 A1 describes a device for introducing drugs into the body during an insufflation. The device described may include a humidity sensor, which in turn is based on a capacitance measurement.

U.S. Pat. No. 5,483,414 describes an impedance sensor for measuring physical parameters, in particular temperature.

The present invention is intended to overcome the disadvantages of the solutions known in the prior art and to provide an insufflation device capable of measuring the humidity of a humidifying material without the need for additional sensors.

### SUMMARY OF THE INVENTION

The invention teaches an embodiment of a heating tube with which the permittivity of the humidifying material can be measured. The basic principle of the solution according to the invention is a construction of the heating tube analogous to a capacitor.

In electrical engineering, capacitors are known as passive components. In principle, capacitors consist of two electrically conductive surfaces (electrodes) that are separated from each other by an insulating material, the dielectric. Plate-shaped electrodes (capacitor plates) are common designs of such capacitors. The electrical properties of the capacitors are determined not only by the surface area, volume and distance between the electrodes, but also by the permittivity of the dielectric between the plates.

It is also well known that a capacitor forms a resistance when an alternating current is applied. This AC resistance is also called impedance and can be described as a complex AC resistance. The impedance can be measured by methods described in the prior art, in particular by measuring the resistance of an applied alternating current.

The description of the capacitor as a “complex resistance” is necessary because the resistance calculated according to formula (I)

$$R = \frac{U}{I} \qquad ( I )$$

is time-dependent. The necessary calculation methods are described in detail in the textbooks of electrical engineering.

It is relevant to the present invention that the impedance of a capacitor or the complex resistance changes by changing the permittivity ε of the dielectric.

In this case, the dielectric consists primarily of the humidifying material and its water content (“moisture content”). Due to the electrical conductivity of water, the electrodes must be electrically isolated from each other. For this purpose, at least one of the electrodes is coated with an electrical insulation.

For the application purpose according to the invention, namely the measurement of the humidity inside an insufflation tube, which in turn contains a heating element and a moist humidifying material, in the simplest case two wires can be arranged as electrodes forming a capacitor as described above. The wires can be arranged within the lumen of the tube, and in particular the wires can be fixed (e.g., glued or welded) to the inner wall of the tube. Fixation is recommended because otherwise the impedance may change with the possible movement of the heating tube. Other possibilities are to integrate the wires into the tube wall (e.g., by molding) or to attach them to the outer wall of the tube (e.g., by gluing). An arrangement within the tube is preferred. The wires can be arranged in a largely straight line (parallel to the orientation of the tube). Alternatively, they can be arranged helically along the tube wall. It is essential for the function of the invention that the humidifying material is located between the wires. In this way, the two wires with opposite poles, at least one of which must be insulated, form a capacitor whose impedance (at constant frequency) depends on the properties of the dielectric. When the moisture content of the dielectric is changed, there are changes in the impedance that can be measured.

### BRIEF DESCRIPTION OF THE DRAWINGS

FIG. 1 provides three views of a insufflation tube which has been constructed in accordance with an embodiment of the present invention in various stages of assembly;

FIG. 2 provides several views of an insufflation tube which has been constructed in accordance with an embodiment of the present invention in which the wires forming the capacitor are formed by the connecting cable of the temperature sensor;

FIG. 3 provides several views of an alternative embodiment of an insufflation tube which has been constructed in accordance with the present invention in which one of the wires forming the capacitor is formed by a heating wire; and

FIG. 4 provides a graphical representation of an exemplary measurement cycle of impedance over time with water or saline being used as the humidifying solution.

### DETAILED DESCRIPTION OF PREFERRED EMBODIMENTS

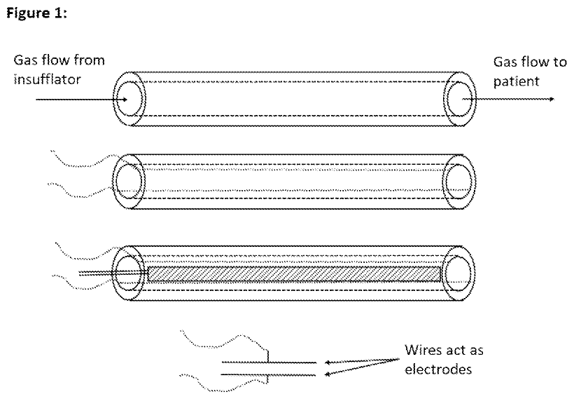

FIG. 1 schematically shows the structure of an insufflation tube according to the invention: An empty tube is shown at the top. Two wires are inserted into the tube and attached to the tube wall (middle). The wires act as electrodes. The wires (electrodes) are electrically insulated from each other and form a capacitor with each other and with the medium between them. The connections to the insufflator are shown. The humidifying material with the heating wire wrapped-around it is shown in the figure below. The electrical connections for the heating wire are also shown.

The insufflation tube according to the invention is typically connected to the insufflator in a dry state, and the insufflator first measures the impedance in a dry state, i.e., with dry humidifying material. The humidifying material is then humidified with the appropriate amount of humidifying material (e.g., water). The amount of water required for this depends on the amount of humidifying material in the tubing. The tubing may have a water fill port. After humidification, the impedance is measured again. During surgery, the humidifying material releases water into the air. The impedance then returns to the level of the dry tube.

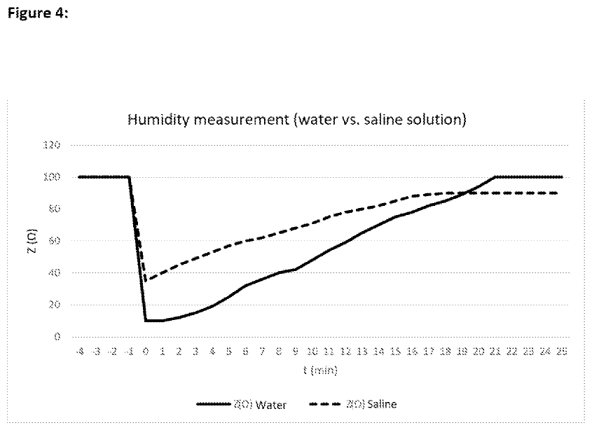

Research has shown that simple metal (e.g., copper) wires with a diameter of 0.1-1 mm are already sufficient to measure the change in the dielectric properties of the humidifying material (see below). FIG. 4 schematically shows the measured change in impedance as a function of the insufflation time.

It has been found that production-related variations in the wires (e.g., wire diameter, length) or their routing can result in small changes in impedances. Since impedance is frequency dependent, such variations can be compensated for by frequency changes. In a particular embodiment of the invention, the impedances are measured by the insufflator in a dry and/or wet state and adjusted to a predetermined nominal impedance by changing the frequency of the applied alternating current.

In an alternative embodiment of the invention, the impedances are measured after manufacture in a dry and/or wet state and the measured values are stored on a data carrier. The stored data may include the following:
Impedance (with dry humidifying material) at one or more specific measurement frequencies;
Impedance (with wet humidifying material) at one or more specific measurement frequencies;
Measuring frequency to obtain a given impedance with wet humidifying material;
Measurement frequency to achieve a given impedance with dry humidifying material.

The data can be stored, for example, on an RFID chip, magnetic tape, or bar code, and the insufflator must have a compatible reader. In this case, the insufflator would read the stored data during or after attaching the insufflation tube and use it to adjust the device.

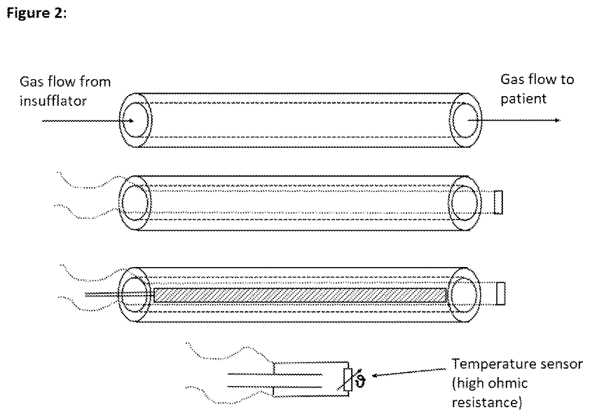

In another embodiment of the invention, the wires forming the capacitor are formed by the connecting cable of a temperature sensor. In this embodiment, a common temperature sensor (e.g., DS18S20) is placed in the tube. In any case, it must be a high-impedance temperature-dependent resistor at the AC frequency used for measurement, so that the capacitor is not short-circuited. A basic circuit diagram is shown in FIG. 2. In this embodiment of the invention, the temperature and the moisture content cannot be measured simultaneously; these properties are measured one after the other, especially in continuous alternation. By applying a direct current to the two connecting cables, the resistance of the temperature sensor can be measured, from which the temperature is then determined. By applying a high-frequency alternating current, it is possible to measure the impedance of the capacitor formed, which is related to the humidity of the humidifying material.

The advantage of this embodiment is that no additional humidity sensor is required. At the same time, the number of wires in the tube is limited. Again, in this embodiment, the lead wires for the temperature sensor can be placed outside or inside the tube or inside the tube wall.

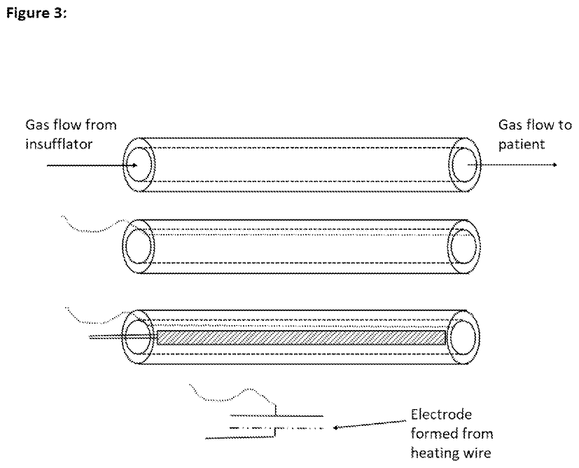

In another alternative embodiment of the invention, one of the wires forming the capacitor may also be a heating wire (FIG. 3). Insufflation tubes with heating wires have been described, for example, in WO 2014/111083 A1. In this embodiment, too, only a temporally alternating heating or measurement is possible: By applying a direct current to the heating wire, it develops heat via the ohmic resistance, which heats the gas passing through and the humidifying material. Again, the advantage already described above is realized that no additional component needs to be introduced into the tube, so that the measurement can be made by using the components already present. By applying a high-frequency AC voltage to the heating wire on the one side and to a parallel wire on the other side, the impedance of the resulting capacitor can be determined and from this, the humidity of the humidifying material can be determined.

In a preferred embodiment of the invention, the gas flow into the insufflation tube is through a humidifying material formed as a braided tube, which in turn is positioned within the insufflation tube. As described in WO 2014/111083 A1, the braided tube may also be wrapped with the humidifying material and the heating wire. In either case, the gas flowing through the braided tube exits through the pores of the sheath surface and is both heated and humidified. In one embodiment of the invention using a metallic braided tube, the increase in capacitor area can positively affect the quality of the measurement.

If the heating wire is made of a material whose resistance increases with temperature (e.g., a material with a positive or negative temperature coefficient (PTC or NTC), as described in WO 2014/111084 A1), then the temperature can also be measured by measuring the resistance of the heating wire. The details of such a temperature measurement are described in WO 2014/111084 A1, so that reference can be made thereto. In such an embodiment of the invention, heating periods, measuring periods for measuring the humidity of the humidifying material, and temperature measurement alternate periodically. The use of digital measuring sensors in such an arrangement is less recommendable, since the supply lines for digital temperature sensors as a bus system are susceptible to interference due to the irradiation of RF signals.

The measurement of the humidity of the humidifying material according to the invention is carried out in a basically known manner, namely by measuring the impedance by means of an applied alternating current of the highest possible frequency (e.g., 100 kHz to 100 MHz). Since the impedance depends on the exact construction of the device according to the invention (among other things, the distance between the wires forming the capacitor, the type of humidifying material, the composition of the humidifying material, the geometrical arrangement of the wires, etc.), an impedance measurement must be carried out for each construction of such a tube. For this purpose, the tube construction shown below is first realized without humidifying material (water), and the impedance of the formed capacitor is measured by the usual methods. Then, the humidifying material is maximally humidified by adding the humidifying material (water). A dummy is then used to heat the tube and allow gas to flow through it. During this process, the impedance is measured continuously or periodically and the impedance curve is recorded. Due to the possible different designs of such insufflation tubes according to the invention, the absolute value of the impedance is comparatively unimportant. What is decisive is the course of the impedance in relation to the degree of humidification of the humidifying material.

FIG. 4 shows an example of the measurement cycle and the results: The sample tube is first measured dry and shows an impedance of 100 ohms. By humidifying it with 10 ml of water, the impedance drops to 10 ohms. By passing a gas (here: CO₂) through the sample and heating it to 39° C., the water evaporates with time or with the gas flow (here: constant gas flow of 10 l/min). After 200 liters have flowed through, the water is practically all used up, the humidifying material is dry again, so that the original impedance of 100 ohms is measured again (approximately). It can be seen that the measured absolute values are different when an isotonic saline solution is used instead of (distilled) water. However, the shape of the measurement curve is similar, so that a humidity measurement is possible in this case as well.

The primary objective of the present invention is to determine the condition of the humidifying material with respect to its water content, i.e., the water content of the humidifying material, without implementing the disadvantages mentioned at the beginning. The primary objective is to generate a replenishment alarm/signal, i.e., a signal informing the user when a replenishment of water is required.

For example, an alarm signal can be triggered when the water content of the humidifying material drops below a preset threshold. For example, the alarm signal can be triggered when a preset threshold value is 50%, 40%, 30%, 20%, 10%, or 5% of the maximum humidity.

In the context of the present invention, the terms “water content of the humidifying material” and “humidity of the humidifying material” are considered synonymous.

FIG. 1 schematically shows the structure of an insufflation tube according to the invention: An empty tube is shown at the top. Two wires are inserted into the tube and attached to the tube wall (middle). The wires, which are electrically insulated from each other, act as a capacitor whose impedance depends on the medium between them. The connections to the insufflator are shown. The humidifying material with the heating wire wrapped around it is shown in the figure below. The electrical connections for the heating are also shown.

FIG. 2 shows an embodiment of the invention in which the wires forming the capacitor are formed by the connecting cable of a temperature sensor.

FIG. 3 shows an alternative embodiment of the invention in which one of the wires forming the capacitor is formed by a heating wire. In an optional variant, the temperature can also be measured by the heating wire (analogous to WO 2014/111083 A1).

FIG. 4 shows an example of the measurement cycle and the results: The sample tube is first measured in dry form and shows an impedance of 100 ohms. At time t-O s, it is humidified with 10 ml of distilled water and the impedance drops to 10 ohms. By passing a gas (here: CO₂) through the sample and heating it to 39° C., the water evaporates with time or with the gas flow (here: constant gas flow of 10 l/min). After 200 liters have flowed through, the water is practically all used up, the humidifying material is dry again, and the original impedance of 100 ohms is measured again (approximately). It can be seen that the measured absolute values are different when an isotonic saline solution is used instead of (distilled) water (dashed line). However, the shape of the measurement curve is similar, so that a humidity measurement is also possible in this case.

1. An insufflation device for use in medical technology comprising:
an insufflator for gas supply and;
an insufflation tube, the insufflation tube including a humidifying material in its interior, the humidifying material being in contact with a heating element, the heating element including a wire which can be activated by applying a current, and
wherein the insufflation tube further includes two mutually insulated wires which together form a capacitor whose impedance depends on a humidity of the humidifying material.

2. The device according to claim 1, wherein at least one of the mutually insulated wires is arranged on an outer wall of the insufflation tube, an inside the wall of the insufflation tube, on an inner wall of the insufflation tube, or inside the insufflation tube.

3. The device according to claim 1, wherein the insufflation tube further includes a temperature sensor.

4. The device according to claim 2, wherein the temperature sensor is disposed at a patient end of the insufflation tube.

5. The device according to claim 3, wherein at least one of the mutually insulated wires forming the capacitor is formed by a connecting cable of the temperature sensor.

6. The device according to claim 1, wherein at least one of the mutually insulated wires forming the capacitor is formed by a heating wire.

7. The device according to claim 1, wherein at least one of the mutually insulated wires forming the capacitor is formed by a metallic braided tube.

8. A method for measuring a water content of a humidifying material located in an insufflation tube of an insufflator device according to claim 1 through which a gas flows, the method including the steps of:
a) applying a high-frequency voltage to the two mutually insulated wires forming the capacitor,
b) determining the impedance of the capacitor, and
c) determining the humidity of the humidifying material from the impedance.

9. The method further comprising the steps of:
a) applying a high-frequency voltage to the two mutually insulated wires forming the capacitor,
b) determining the impedance of the capacitor with dry humidifying material,
c) humidifying the humidifying material,
d) determining the impedance of the capacitor with humidified humidifying material,
e) passing gas through the insufflation tube, and
f) determining a humidity of the humidifying material from a change in impedance during insufflation.

10. The method according to claim 8, wherein an alarm signal is triggered when the humidity of the humidifying material falls below a preset threshold value.

11. The method according to claim 10, wherein the preset threshold value corresponds to 50%, 40%, 30%, 20%, 10% or 5% of the maximum humidity.

12. The method according to claim 8, wherein the impedance of the capacitor is measured before gas is passed through the insufflator with dry and/or wet humidifying material by applying a high-frequency voltage and by changing a frequency to a predetermined nominal impedance with dry and/or wet humidifying material is set, the frequency at which the nominal impedance is reached being used for the intended insufflation with gas humidification.

13. The method according to claim 8, wherein the impedance of the capacitor after manufacture with dry and/or wet humidifying material is measured by applying a high-frequency voltage and by changing the frequency a predetermined nominal impedance with dry and/or wet humidifying material is set, the frequency at which the nominal impedance is reached being stored on a data carrier, the data carrier being read out by the insufflator, and the stored data being used for the intended insufflation with gas humidification.
